(19) United States
(12) Patent Application Publication
(10) Pub. No.: US 2005/0236376 A1
(43) Pub. Date: Oct. 27, 2005

(54) Energy generation

(76) Inventor: Christopher Robert Eccles, Colchester (GB)

Correspondence Address: MCANDREWS HELD & MALLOY, LTD, 500 WEST MADISON STREET, SUITE 3400, CHICAGO, IL, 60661, US

(21) Appl. No.: 11/061,048

(22) Filed: Feb. 18, 2005

Related U.S. Application Data
(63) Continuation of application No. 09/830,040, filed on Aug. 13, 2001.

(51) Int. Cl. A61N 1/18; B23K 9/00

(52) U.S. Cl. 219/121.36; 373/22

(57) ABSTRACT

Methods and apparatus are described for releasing energy from hydrogen and/or deuterium atoms. An electrolyte is provided which has a catalyst therein suitable for initiating transitions of hydrogen and/or deuterium atoms in the electrolyte to a subground energy state. A plasma discharge is generated in the electrolyte to release energy by fusing the atoms together.

The present invention relates to the generation of energy, and more particularly to the release of energy as a result of both a state-transition in hydrogen and fusion of light atomic nuclei.

Normally, fusion processes are able to be initiated only at extremely high temperatures, as found in the vicinity of a nuclear fusion (uranium or plutonium) detonation. This is the principle of most thermonuclear bombs. Such a release of energy is impractical as a means of providing the power to generate electricity and heat for distribution, as it occurs too rapidly with too high a magnitude for it to be manageable.

In recent years, many attempts have been made to initiate controlled fusion processes at high temperatures by the enclosure of a region of plasma-discharge within a confined space, such as a toroidal chamber, using electromagnetic restraint. Such attempts have met with little commercial success to date as systems which employ such a technique have so far consumed more energy than they have produced and are not continuous processes.

Another approach which has been attempted in order to achieve fusion of light nuclei has been the so-called “cold fusion” technique, in which deuterium atoms have been induced to tunnel into the crystal lattice of a metal such as palladium during electrolysis. It is claimed that the atoms are forced together in the lattice, overcoming the repulsive electrostatic force. However, no clear and unambiguous demonstration of successful cold fusion has yet been presented publicly.

The present invention provides a method of releasing energy comprising the steps of providing an electrolyte having a catalyst therein, the catalyst being suitable for initiating transitions of hydrogen and/or deuterium atoms in the electrolyte to a sub-ground energy state, and generating a plasma discharge in the electrolyte. The applicants have determined that this method generates substantially more energy than the power input used to generate the plasma, whilst doing so in a controllable manner.

Preferably, the plasma discharge is generated by applying a voltage across electrodes in the electrolyte and an intermittent voltage has proved particularly beneficial in increasing the level of energy generation. It also provides a means of controlling the process to maintain a consistent level of energy production over a significant period of time.

The application of a voltage higher than that necessary to generate plasma is also beneficial to the process and will be typically in the range 50V to 20000V and preferably between 300 and 2000V, but may be higher than 20000V, whereas in conventional electrolysis techniques low voltages of about 3 volts are used and applied continuously across the electrodes.

The applied voltage may be DC or provided at a switching frequency of up to 100 kHz. The duty cycle of the applied voltage is preferably in the range 0.5 to 0.001, but may be even lower than 0.001. During the pulse period a monomolecular layer of metal hydride may be formed at the cathode-Helmholtz layer interface and subsequently decays to form gas in the nascent state comprising monatomic hydrogen and/or deuterium. The waveform of the applied voltage may be substantially square shaped. Whilst application of DC to the electrode does produce the metal hydride and monatomic hydrogen and/or deuterium, the use of a pulsed voltage has been found to be more efficient as most dissociation of the hydride then occurs between the pulses.

In applications where the electrolyte is flowed past the electrodes it may be preferable to use two separate cathodes, the first of which will be engineered to optimise production of hydrogen/deuterium atoms and the second of which will provide the plasma discharge. In this instance the direction of flow of the electrolyte is from first to second cathode. The design of the apparatus seeks to direct the flow of electrolyte to maximise contact of monatomic hydrogen or deuterium atoms with the plasma. The characteristics and magnitudes of the voltages applied to each cathode are preferably similar, but may have different duty periods.

In a preferred embodiment, the cathode design and applied voltage are such as to provide a current density of 400,000 amps per square meter or even greater. More preferably, the current density at the cathode is 500,000 amps per square meter or above.

In carrying out a preferred method in accordance with the invention, it has been found that the process may be assisted by initial heating of the electrolyte, which may be water or a salt solution, prior to applying electrical input to the vessel. A temperature in the range 40 to 100° C., or more preferably 40 to 80° C., has been found to be particularly beneficial.

The ratio of water to deuterium oxide (D₂O) in the electrolyte may be varied to control the energy generation. In some circumstances it may be preferable to use “light” water H₂O alone and in others to use D₂O alone. Additionally, the amount of catalyst added to the electrolyte may be varied as a controlling factor and preferably lies in the range 1 to 20 mMol.

In preferred embodiments, the method includes the step of generating a magnetic field in the region of the electrodes. The intensity and/or frequency of the current used to generate the field may be adjusted to move the plasma discharge away from the electrode from which it is struck in order to minimise erosion and extend the operating life of the system. Only slight separation may be required to achieve this effect.

In further preferred embodiments, the heat generated by the process may be removed and utilised by way of a number of known and proven technologies including the circulation of the electrolyte through a heat exchanger, or using heat pipes to produce heating, or alternatively to produce electricity using a pressurised steam cycle or a low-boiling-point fluid turbine cycle, or by other means.

The present invention further provides apparatus for carrying out methods disclosed herein comprising an anode, first and second cathodes, a reaction vessel having an inlet and an outlet, means for feeding an electrolyte through the vessel from its inlet to its outlet, the electrolyte having a catalyst therein suitable for initiating transitions of hydrogen and/or deuterium atoms in the electrolyte to a sub-ground energy state, means for applying a voltage across the anode and the first cathode to form hydrogen and/or deuterium atoms, and means for applying a voltage across the anode and second cathode to generate a plasma discharge in the electrolyte, the second cathode being downstream from the first cathode.

During the methods described herein, atoms of hydrogen and/or deuterium are believed to undergo a fundamental change in their structure by exchange of photons with salts in solution. The applicants believe that this change, and the observed phenomena, can be explained as set out below.

It is well known that a system comprising a spherical shell of charge (the electron path) located around an atomic nucleus constitutes a resonant cavity. Resonant systems act as the repository of photon energy of discrete frequencies. The absorbtion of energy by a resonant system excites the system to a higher-energy state. For any spherical resonant cavity, the relationship between a permitted radius and the wavelength of the absorbed photon is:
2πr=nλ
where n is an integer
and λ is the wavelength

For non-radiating or stable states, the relationship between the electron wavelength and the allowed radii is:
2π[nr₁]=2πr₍ₙ₎=nλ₍₁₎=λ₍ₙ₎ (2)
where
n=1
   or
n=2, 3, 4 . . .
   or p1 n=½, ⅓, ¼
   and
λ₍₁₎=the allowed wavelength for n=1
r₍₁₎=the allowed radius for n=1

In a hydrogen atom (and the following applies equally to a deuterium atom), the ground state electron-path radius can be defined as r_{(O)}. This is sometimes referred to as the Bohr radius, a_{O}. There is normally no spontaneous photon emission from a ground state atom and thus there must be a balance between the centripetal and the electric forces present. Thus:
[m₍ₑ₎.v₁²]/r_{(O)}=Ze²/(4π.ε_{(O)}.r_{(O)}²) (3)
where
m₍ₑ₎=electron rest mass
v₁=ground state electron velocity
e=elementary charge
ε_{(O)}=electric constant (sometimes referred to as the permittivity of free space)
Z=atomic number (for hydrogen, 1)

Looking first at the excited (higher energy) states, where the hydrogen atom has absorbed photon(s) of discrete wavelength/frequency (and hence energy), the system is again stable and normally non-radiating, and to maintain force balance, the effective nuclear charge becomes Z_{eff}=Z/n, and the balance equation becomes:
[m₍ₑ₎.vₙ²]/nr_{(O)}=[e²/n]/(4π.ε_{(O)}.[nr_{(O)}]²) (4)
where
n=integer value of excited state (1, 2, 3 . . . )
vₙ=electron velocity in the nth excited state

The absorbtion of radiation by an atom thus results in an excited state which may decay to ground state, or to a lower excited state, spontaneously, or be triggered to do so, resulting in the re-release of a quantum of energy in the form of a photon. In any system consisting of a large number of atoms, transitions between states are occurring continuously and randomly and this activity gives rise to the observable spectra of emitted radiation from hydrogen.

Each value of n corresponds to a transition which is permitted to occur when a resonant photon is absorbed by the atom. Integer values of n represent the absorbtion of energy by the atom.

Fractional values for n are allowed by the relationship between the standing wavelength of the electron and the radius of the electron-path, given by (2), above. To maintain force balance, transitions involving fractional values for n must effectively increase the nuclear charge Z to a figure Z_{eff}, and reduce the radius of the electron-path accordingly. This is equivalent to the atom emitting a photon of energy while in the accepted ground state, effecting a transition to a sub-ground state. Because the accepted ground state is a very stable one, such transitions are rarely encountered but the applicants have discovered that they can be induced if the atom is in close proximity to another system which acts as a “receptor-site” for the exact energy quantum required to effect the transition.

The emission of energy by a hydrogen atom in this way is not limited to a single transition “down” from ground state, but can occur repetitively and, possibly, transitions to ⅓, ¼, ⅕ etc states may occur as a single event if the energy balance of the atom and the catalytic system is favourable. Of course, the usual uncertainty principles forbid the determination of the behaviour of any individual atom, but statistical rules govern the properties of any macroscopic (>10⁹ quanta) system.

When a “ground-state” hydrogen atom emits a photon of around 27 eV, the transition occurs to the a_{O}/2 state as demonstrated above and the effective nuclear charge increases to +2e. A new equilibrium for the force balance is now established. The electron path radius is reduced. The potential energy of the atom in its reduced radius-state is given by
V=−{Z_{(eff)}e²/[4πε_{(O)}/2)]}=−{4×27.178}=−108.7 eV

The kinetic energy, T, of the reduced electron path is given by
T=−[V/2]=54.35 eV

Similarly, it can be seen that the kinetic energy of the ground state electron path is about 13.6 eV. Thus there is a net change in energy of about 41 eV for the transition:
H{Z_{(eff)}=1; r=a_{(O)}} to H{Z_{(eff)}=2; r=a_{(O)}/2}

That is to say, of this 41 eV, about 27 eV is emitted as the catalytic transfer of energy occurs, and the remaining 14 eV is emitted on restablisation to the force balance.

The radial “ground-state” field can be considered as a superposition of Fourier components. If integral Fourier components of energy equal to m×27.2 eV are removed, the positive electric field inside the electron path radius increases by
(m)×1.602×10⁻¹⁹C

The resultant electric field is a time-harmonic solution of the Laplace equations in spherical co-ordinates. In the case of the reduced radius hydrogen atom, the radius at which force balance and the non-radiative condition are achieved is given by
r₍ₘ₎=a_{(O)}/[m+1]
where m is an integer.

From the energy change equations given above, it will be appreciated that, in decaying to this radius from the so-called “ground-state”, the atom emits a total energy equal to
[(m+1)²−1²]×13.59 eV (5)

The applicants have found that such energy emissions as take place according to (5), above, only appear to occur when the hydrogen or deuterium is found in the monatomic (or so-called “nascent”) state. Molecular hydrogen might be made to behave similarly, but the transition is more difficult to achieve owing to the higher energies involved.

In order to achieve the transition in monatomic hydrogen (H) or deuterium (D), it is necessary to accumulate the molecular form in the gas phase on a substrate such as nickel or tungsten which favours the dissociation of the molecule. As well as being dissociated into the monatomic form, the hydrogen or deuterium should be bound to the catalytic system to initiate the reaction. The preferred method of achieving this is by electrolysis using cathode material which favours dissociation.

The applicants have discovered that the catalytic systems which encourage transitions to sub-ground-state energies are those which offer a near-perfect energy couple to the [m×27.2] eV needed to “flip” the atom of H or D. It appears from experiment that the effective sink of energy provided by the catalyst need not be precisely equal to that emitted by the atom. Successful transitions have been achieved when there is an error of as much as ±2% between the energy emitted by the atom and that absorbed by the catalytic system. One possible explanation for this is that, in a macroscopic sized system, although the transitions are initiated by a close match in energy level, such discrepancies as arise are manifested as an overall loss or gain in the kinetic energies of the recipient ionic systems. It is thought that spectroscopic analysis of active H or D catalytic systems may provide evidence of this.

One catalyst that has been found to initiate the transition to the a_{O}/n state is rubidium in the Rb+ ionic species. If a salt of rubidium, such as the carbonate Rb₂CO₃ is dissolved in either water or deuterium oxide (heavy water), a substantial dissociation into Rb⁺ and (CO₃)²⁻ ions takes place. If the Rb⁺ ions are bound closely to monatomic H or D, the transition to the a_{O}/n state is encouraged by the removal of a further electron from the rubidium ion, by provision of its second ionisation energy of about 27.28 eV. Thus:
Rb⁺+H{a_{(O)}/p}+27.28 eV −>
Rb²⁺+e⁻+H{a_{(O)}/[p+1]}+{[(p+1)²−p²]×13.59}eV
where p represents an integral number of such transitions for any given H and D atom and by spontaneous re-association:
Rb²⁺+e⁻=Rb⁺+27.28 eV

Thus, the rubidium catalyst remains unchanged in the reaction and there is a net yield of energy per transition.

Other catalytic systems can be used which have ionisation energies approximating to [m×27.2]eV, such as titanium in the form of Ti²⁺ ions and potassium in the form of K⁺ ions.

The applicants believe that the above explanation is consistent with currently accepted quantum theory as discussed below.

Commencing with the equations of Rydberg and Schrödinger it can be shown that fractional numbers for the quantum energy states in hydrogen yield possible transitions which result in emissions at frequencies which are in accord with observed UV and X-ray spectra. It is therefore possible that the conditions conducive to initiating such transitions may be artificially reproduced in the laboratory under certain circumstances.

The Rydberg formula for the frequency of emitted radiation from a transition in monatomic hydrogen is:
v=R₍ₕ₎c(1/n₍₂₎²−1/n₍₁₎²)
where:

v is the frequency of the emitted photon

R₍ₕ₎ is Rydberg constant, 1.097373 c 10⁷ m⁻¹

c is the speed of light in vacuo, 2.997×10³ ms⁻¹

and

n₍₁₎, n₍₂₎ are the transition states

It can be seen from the above that, if the resultant energy state of the hydrogen atom is that which requires n₍₂₎ to be equal to ½, emissions will occur which are of higher frequency than the observed Lyman 2-1 transition in the ultra-violet at 2.467×1°¹⁵ Hz (about 121 nm). There is, indeed, an observed emission at a wavelength of about 30.8 nm, which appears to be confirmed by recent studies of galactic cluster emissions by Böhringer et al (Scientific American, January 1999) and it is difficult for the inventor to conceive of any other quantum-mechanical event which would give rise to such an emission, other than a transition, in accord with the above theory, from 1 to ½ in nascent hydrogen.

As can be seen from the above use of the standard Rydberg equation, such behaviour of hydrogen in the monatomic state views the conventional hydrogen “ground-state” as one of many stable electronically-preferred states for single H atoms.

To summarise, a proliferation of H or D atoms is produced which may have had significantly diminished electron-path-radii by virtue of exchange of photons with their environment. These atoms appear to be relatively unreactive chemically and appear not to readily take the molecular form H-H or D-D. This is a fortunate property which has significance and enables fusion pathways, as described below.

The fusion of light nuclei, hydrogen and deuterium, to form heavier elements such as helium is one which has traditionally been encouraged by subjecting the reactants to extremes of temperature and pressure. This has been necessary because there is a large electric charge barrier to overcome in order to bring nuclei close enough for fusion to occur.

Using atoms with diminished electron path radius, adjacent nuclei may experience a corresponding reduction in electric barrier and internuclear separations may become smaller. With reductions in internuclear separation, fusion processes become more probable, and more easily occasioned.

There are two principle fusion pathways for deuterium atoms. The first is:
²₁D+²₁D=³₂He+¹₀n
where two deuterium nuclei fuse to produce an isotope of helium and a free neutron, which subsequently decays (half-life 6.48×10²S), with emission of a β⁻ particle of medium energy (about 0.8 Mev), and a type of neutrino, to become a stable proton.

The second is:
²₁D+²₁D=³₁T+¹₁H
where the two deuterium nuclei fuse to produce the isotope of hydrogen known as tritium (T) and a free stable proton. The tritium eventually decays (half-life 12.3 years), with emission of a β⁻ particle of very low energy (about 0.018 MeV), to become ³₂He

Of the two, the second fusion path is preferred for the peaceful exploitation of its energy yield, because the fusion products are (relatively) harmless on production, and decay to completely innocuous species within a short time, emitting radiation which can be effectively shielded by a thin sheet of kitchen foil or by 10 mm of acrylic plastic, for example.

When deuterium nuclei are forced together under high temperature and pressure conditions (as in a thermonuclear bomb), there is a greater than 50% probability for the first pathway to be the dominant one. This is because the high temperature process takes no account of nuclear alignment at the point of fusion. It is actually a matter of forcing nucleic together indiscriminately and hoping that enough fuse to produce an explosion. However, the applicants believe, in accord with established theory, that it is the alignment of the nuclei with respect to the charges in each nucleus which ultimately determines the favourable fusion path.

In order to achieve a higher probability for the second, less hazardous, pathway, the approaching nuclei need to have time to align electrostatically such that the proton-proton separation is at a maximum. This can only be achieved at far lower energies than those found in a thermonuclear bomb. By the use of entities with diminished electron-path-radii, and correspondingly potentially smaller internuclear distances, fusion can be initiated at lower temperatures (and consequently lower energies), allowing for the charge-related alignment necessary to achieve a high probability for the second, tritium-forming, pathway. By introducing deuterium of diminished electron-path-radius into a plasma discharge which is confined within the water in the vessel itself, fusion is may be initiated. Temperatures of the order of 6000 K are obtained within certain plasma discharges and this, coupled with multiple quantum transitions to produce deuterium of diminished electron-path-radius, produces a substantial yield of energy from the two-stage process.

Another possible but less likely fusion pathway for hydrogen atoms is:
¹₁H+¹₁H=²₁D+β⁺+τ
whereby β⁺ is produced as one of the products.

Embodiments of the invention will now be described by way of example and with reference to the accompanying schematic drawings, wherein:

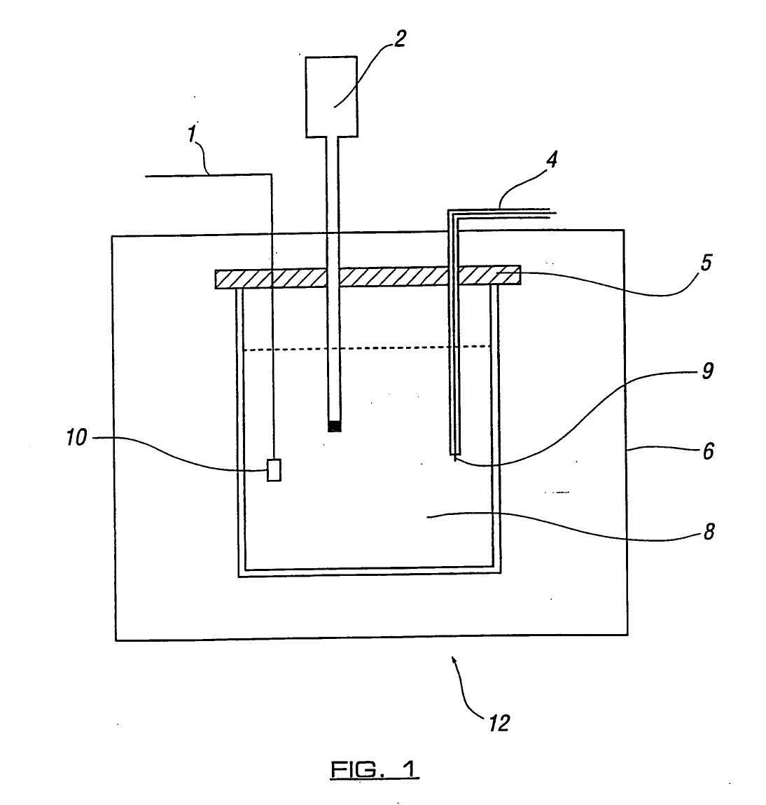

FIG. 1 shows an apparatus for carrying out a method in accordance with the invention on a relatively small scale;

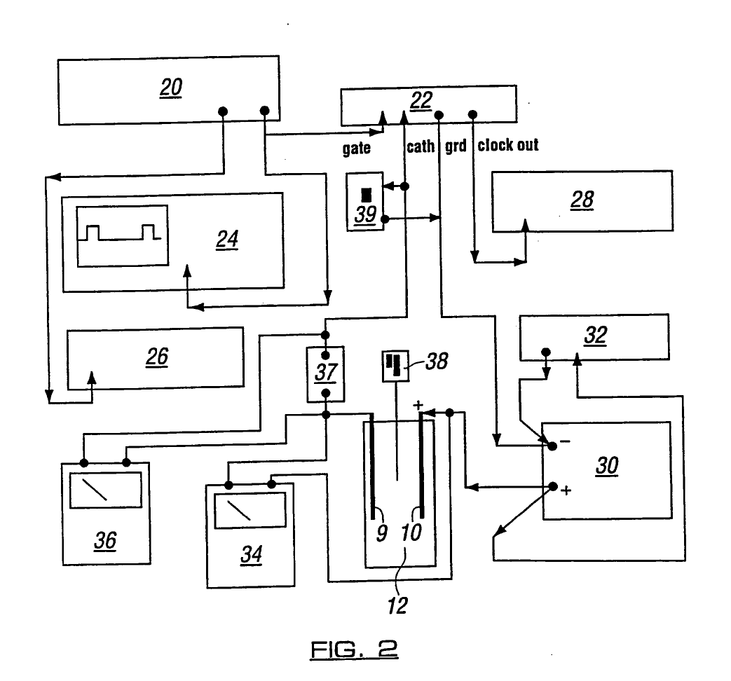

FIG. 2 shows a system for operating and measuring the performance of the apparatus of FIG. 1;

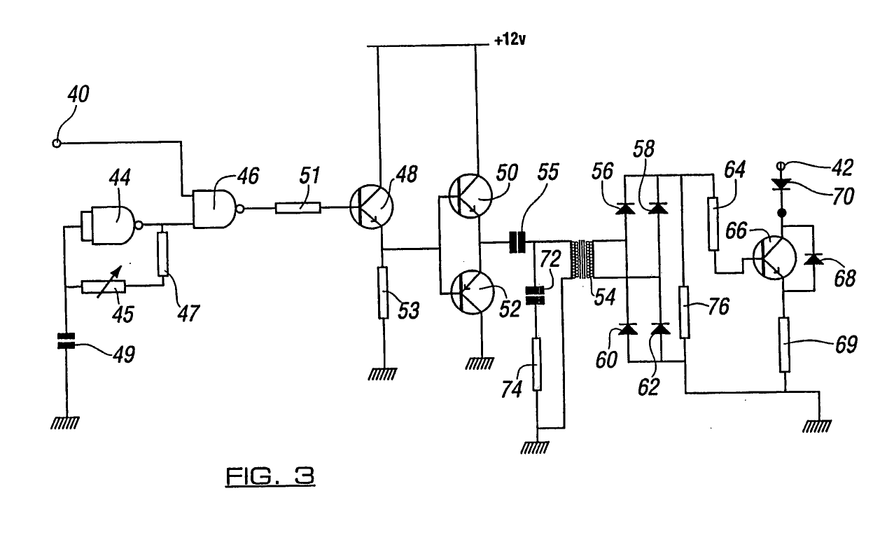

FIG. 3 shows a circuit diagram high voltage, high frequency switching circuit for the system of FIG. 2;

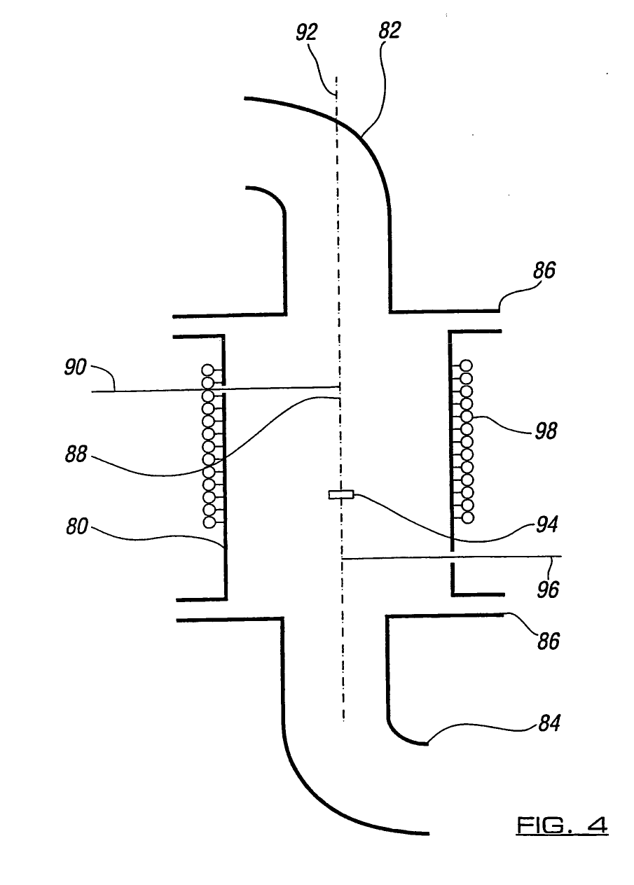

FIG. 4 shows an apparatus for carrying out a method in accordance with the invention on a larger scale than that of the FIG. 1 apparatus; and

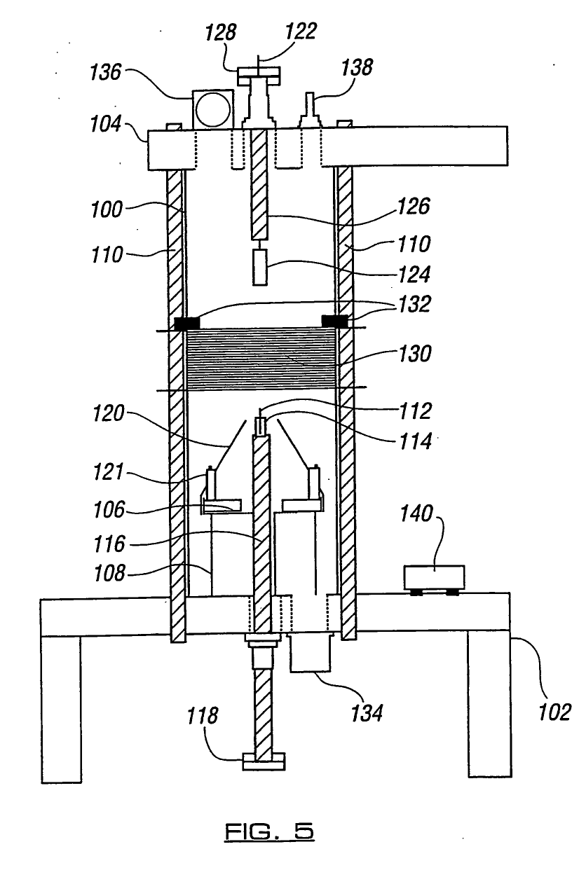

FIG. 5 shows a further apparatus for carrying out a method of the invention which includes two cathodes.

The apparatus of FIG. 1 enables the generation of energy according to the principles of the invention in the laboratory. Any risk of thermal runaway is minimised, whilst demonstrating that the level of energy release from the two stages is far in excess of that which would result from any purely chemical or electrochemical activity. It also enables easy calorimetry, safe ducting away of off-gases, and of subsequent extraction of liquid for titration (to demonstrate that no chemical action takes place during the operation of the apparatus).

A 250 ml beaker is provided with a glass quilt or expanded polystyrene surround 6 to act as insulation. This can include an inspection cut-out so that the area around the cathode 9 can be observed from outside. The beaker contains 200 ml of water, into which is dissolved a small quantity of potassium carbonate so as to give a solution of approximately 2 mMol strength. A platinum lead wire 1 is earthed to the laboratory reference ground plane. The anode 10, a sheet of platinum foil of approximately 10 mm² in area, is attached to this lead wire by mechanical crimping. A digital thermometer 2 is inserted into the liquid in the vessel. A 0.25 mm diameter tungsten wire cathode 9 is sheathed in borosilicate glass or ceramic tube 4 and sealed at the end immersed in the electrolyte so as to expose 10 mm to 20 mm of wire in contact with the liquid. The entire assembly of lead wires and the thermometer is carried by an acrylic plate 5 which enables of easy dismantling and inspection of the apparatus.

A supply of up to 360 volts DC, capable of supplying up to 2 amperes, is arranged external to the described apparatus. The positive terminal of this supply is connected to the laboratory reference ground plane and the negative terminal is connected to one pole of an isolated high-voltage switching unit. The other pole of the switch is connected to the tungsten wire cathode 9 externally of the apparatus.

To operate the apparatus, the solution 8 is initially brought up to between 40° C. and 80° C. either by preheating outside the apparatus or by passing power through a heating element in the solution (not shown). When the solution is between these temperatures it is either transferred to the above apparatus or, if a heating element is used, this is turned off.

With all connections made as described, the switch is set to operate at a duty cycle of 1% and a pulse repetition frequency of 100 Hz. It will be seen through the inspection cut-out that an intense plasma-arc is intermittently struck under the water at or near the cathode. If equipment is available to monitor the current drawn, it will be seen that the system consumes in the region of 1 watt when the switching circuits is operating. It will be seen by the rapid rise in temperature in the apparatus that far more energy is being released than can be accounted for by the electrical input. As a comparison, a heater element can be substituted for the electrodes and operated 1 watt and the effects observed. There is really no need for sophisticated calorimetry to verify that large quantities of energy are being released close to the cathode of the equipment, such is the magnitude of the reaction for the process, as compared to a test with a resistive heating element of the same input power.

The data obtained from a representative one-hour session with this apparatus as shown as Table 1, below:

Pre Run Measurements

| | | |
|---|---|---|
| Commencing volume of electrolyte | 0.200 | l |
| Commencing temperature of cell | 39.200° | C. |
| Laboratory ambient temperature | 20.500° | C. |
| Spec. heat capacity of vessel | 70.300 | J · ° C.⁻¹ |
| Spec. heat capacity of electrolyte | 4180.000 | J · I⁻¹ · ° C.⁻¹ |
| Steady RMS voltage | 4.000 | volts |
| Steady RMS current | 0.067 | Amps |

Post Run Results

| | | |
|---|---|---|
| Duration of input | 3600.000 | secs |
| Final volume of electrolyte | 0.180 | l |
| Final temperature of cell | 93.600° | C. |
| Steady RMS voltage | 6.700 | volts |
| Steady RMS current | 0.122 | Amps |
| Time-averaged power in | 0.506 | watts |

Results Summary

| | | |
|---|---|---|
| Vessel Gain | 3824.320 | Joules |
| Electrolyte gain | 43181.740 | Joules |
| Radiated power | 38681.030 | Joules |
| Evaporated loss | 48509.240 | Joules |
| TOTAL ENERGY IN | 1820.070 | Joules |
| TOTAL ENERGY OUTPUT | 134196.300 | Joules |

It can be seen from this table that the total energy input during this test was measured at 1820 Joules and, taking as a rough guideline that 200 ml of water requires the input of 838 joules of energy to raise it by 1° C., then by direct heating the water would be expect to rise by some 2° C., bearing in mind radiative losses. In fact, during the experiment the water temperature was raised from 39.2° C. to 93.6° C. and considerable steam was also liberated. Furthermore, the calculated energy output of 134196 Joules does not take account of secondary effects such as light-energy output and Faradaic electrolysis.

A system suitable for operating the apparatus of FIG. 1 is illustrated in a block diagram in FIG. 2. A pulse generator 20 supplies a variable duty-cycle pulse waveform to a high voltage switch unit 22. The pulse waveform may be monitored on an oscilloscope 24 and its repetition frequency is displayed on a first frequency counter 26. A second frequency counter 28 is provided to monitor the clock speed of the switch unit 22. Power supply 30 is operable to apply a voltage between 0 and 360 V to an electrode of the apparatus 12, shown in FIG. 1. The voltage level may be read from a digital multimeter 32. The RMS voltage across the electrodes 9 and 10 is indicated on a multimeter 34 and the RMS current passing between the electrodes is shown on another multimeter 36, by measuring the voltages developed across a 1 ohm resistor 37. The temperature in the apparatus 12 is indicated on a dip temperature probe 38. The switch unit 22 may be bypassed by a push button switch 39 to apply a constant voltage across the electrodes.

A circuit diagram of the switch unit 22 is shown in FIG. 3. In the system of FIG. 2, input 40 is connected to the output of pulse generator 20. The output 42 of the switch unit is connected to the cathode of the apparatus 12. Two NAND gates 44 and 46 are two fourths of a Schmitt-trigger 2 input NAND gate chip type 4093. NAND gate 44 operates as an astable multivibrator, with its repetition frequency set by a preset resistor 45. The output of gate 44 is fed to one input of NAND gate 46, the other input forming circuit input 40. The output of NAND gate 46 is connected to a three transistor amplifier consisting of transistors 48, 50 and 52. The amplifier is in turn connected to one end of the primary of a transformer 54, the other end being connected to earth. The transformer output is fed to a bridge rectifier formed from diodes 56, 58, 60 and 62.

The rectifier output is fed via a resistor 64 to the gate of an insulated gate bipolar transistor 66 (IGBT). The load of the apparatus 12 is connected in the drain circuit of the IGBT. A 15 kV diode 68 is connected between the drain and the source of the IGBT 66 to protect the IGBT from the sizeable EMI emissions from plasma discharges in the apparatus 12 and avoids damage to this sensitive semiconductor. A further diode 70 is provided between the drain of the IGBT and the circuit output 42 to act as an EMI blocker in a similar way. A standard 20 mm 5A quick-blow fuse 69 is connected between the source of the IGBT and ground in order to protect the device against overcurrent.

The operation of the circuit of FIG. 3 is as follows. The repetition frequency is NAND gate 44 is preferably set to between 4 and 6 MHz. Pulse generator 20 is adjusted to set the duty of the switching. On receipt of an external pulse from the generator, NAND gate 46 passes a packet of 4 to 6 MHz square waves to the amplifier. The amplifier has considerable current gain and enables the primary of the transformer 54 to be driven resonantly with the RC circuit formed by capacitor 72 and resistor 74 which are connected in parallel therewith. The transformer 54 has a step-up ratio of 2:1 and a 4 to 6 MHz signal of approximately 19 volts appears across the bridge rectifier. The impedance of the rectifier output is essentially determined by a parallel resistor 76, such that the switch-on and switch-off time of the IGBT 66 is very fast. Thus, there is never a point in the operation of the device when it is dissipating any measurable power. The load of the apparatus 12 is placed in the drain circuit of the IGBT, which is therefore operating in “common-source” made to ensure that its source terminal never rises above high-side ground potential. This, again, is a configuration which uses excess input power. This circuit ensures a rise time of the switched waveform which is less than 10 nS and a fall time which can be as low as 30 nS at modest supply voltages.

Preferred component values and types for the circuit of FIG. 3 are as follows:
Transistor 4, 50—2N 3649
Transistor 52—2N 3645
Diodes 56, 58, 60, 62—BAT85 Schottky
Transformer 54—RS195-460
IGBT 66—GT8Q101
Diode 68—15 kv EHT

Diode 70—1N1198A

| Resistor | Value (Ω) | Capacitor | Value |
|---|---|---|---|
| 47 | 1.8k | 49 | 10 pF |
| 51 | 33 | 55 | 33 nF |
| 53 | 220 | 72 | 22 pF |
| 74 | 56 | | |
| 76 | 560 | | |
| 64 | 56 | | |

A second apparatus for carrying out the invention is illustrated in FIG. 4. This apparatus comprises a tubular chamber 80, which may be constructed from a nonmagnetic metal or metal alloy material such as, but not exclusively, aluminium or Duralumin, or may alternatively be constructed from a non-permeable ceramic material or from borosilicate glass. The tubular chamber 80 is constructed in flanged form to allow of its incorporation into a system of pipework via flanges 82 and 84 and gaskets 86. Entering the chamber 80 are two electrodes, the cathode 88 being sheathed in an insulating glass or ceramic tube 90 and shaped so as to present itself along the axis of the chamber 92. The anode 94 is connected to a similar insulated wire 96 and is shaped so as to present a circular plate opposite the cathode 88. The distance between the cathode tip and the anode plate should be approximately equal to the radius of the chamber 80. The cathode may be constructed from tungsten, zirconium, stainless steel, nickel or tantalum, or any other metallic or conductive ceramic material which may contribute to, or occasion, the dissociative process described above. The anode may be constructed from platinum, palladium, rhodium or any other inert material which does not undergo any significant level of chemical interaction with the electrolyte.

Surrounding the chamber 80, and concentric with it, is a winding 98 of enamelled copper or silver wire of diameter 0.1 to 0.8 mm consisting of up to several thousand turns of the wire. The purpose of this winding 98 is to create an axial magnetic field inside the chamber 80.

Electrolyte comprising deuterium oxide, in combination with ordinary “light” water in varying proportions, and containing high-molarity salts of, but not exclusively of, potassium, rubidium or lithium, or combinations of such salts, is pumped through the chamber 80, in a direction such that the anode is downstream of the cathode.

The anode lead wire 96 is connected to the ground plane or zero volts. The cathode 88 is connected to a variable source of between 50 and preferably 2000 volts negative with respect to the grounded anode 94, but may be coupled to a voltage of up to several tens of thousands of volts negative with respect to such anode 94. To enhance performance of the invention, the negative voltage may be supplied in the form of pulses having a duty cycle between 0.001 and 0.5.

The winding 98 is energised with an alternating voltage such as to provide a current flow of typically between 0.5 and 1.5 amps initially. The frequency of the applied alternating voltage should be variable from DC up to 15 kHz and may, in addition, be synchronous with pulses applied to the cathode 88.

Under these conditions, a plasma arc will strike close to the cathode 88. The intensity and frequency of the current flowing in winding 98 may be adjusted to provide for the removal of the plasma arc from the immediate vicinity of the cathode 88 to avoid excessive evaporation of the material from the cathode 88.

The volume of electrolyte pumped through chamber 80 and past the plasma arc may be varied such as to stabilise the temperature of such electrolyte in a closed system at below at its boiling point.

Heat may be extracted from the electrolyte by passing it through a heat exchanger before its re-introduction into the chamber 80. Provision may be made to top-up the water/deuterium content of the electrolyte as this becomes depleted by operation of the apparatus. The system may operate at a range of pressures to facilitate heat removal.

A further apparatus for carrying out the invention, similar to that of FIG. 4, is shown in FIG. 5 on a scale of approximately 1:2.5. It comprises a borosilcate reaction tube 100 supported at one end on a machined nylon support bridge 102. A second machined nylon element 104 is mounted across the other end of the tube. The bridge 102 and element 104 are clamped against the tube 100 by 8 mm threaded stainless steel studs 110.

A first cathode 106 is in the form of a nickel wire mesh. It is mounted towards one end of tube 100 on a stainless steel support 108. Electrical connection to the first cathode 106 is via a PVC-sleeved wire (not shown).

A second cathode 112 consists of an 0.5 mm diameter length of tungsten wire provided within a drilled macor ceramic sheath 114, which is in turn placed within a 10 mm stainless steel tube 116. Tube 116 passes through the support 102 and has a perspex end cap 118 on the external end through which the second cathode 112 passes. A PVC funnel 120 is provided around the-second cathode and is tapered towards it, with the cathode tip adjacent the narrower open end thereof. The funnel is supported on sleeves 121 provided on the stainless steel support 108.

The anode comprises an 0.25 mm diameter platinum wire 122 which is connected at one end within the tube 100 to a sheet of platinum foil 124. Like the second cathode 112, the anode is provided within a 10 mm diameter stainless steel tube 126, which passes through nylon element 104 and is closed at its external end by a perspex end cap 128. Platinum wire 122 passes through the end cap 128.

A plasma deflection coil 130 is mounted within tube 100 between the anode 124 and cathodes 106, 112. Electrical power is fed to the coil via connectors 132.

Electrolyte is supplied to the tube 100 via a brass inlet 134 provided through the support bridge 102 and flows out through nylon element 104 via a brass outlet 136. An additional brass outlet 138 is also provided in nylon element 104 to allow the electrolyte to be sampled during operation of the apparatus. Fuse holders and cable connectors for the apparatus are provided in a unit 140 mounted on the support bridge 102.

The apparatus of FIG. 5 is operated in a similar manner to that of FIG. 4, as discussed above. The primary distinction is that two cathodes 106, 112 are employed in place of a single cathode. In use, electrolyte is fed through the tube 100, past the electrodes, from inlet 134 to outlet 136. A pulsed voltage is applied to the first cathode 106 such that a layer of metal hydride is formed on it surface during the voltage pulses and subsequently dissociates to form nascent monatomic hydrogen/deuterium. The applied voltage characteristics are selected to optimise the production rate of the monatomic hydrogen/deuterium. These products are channelled towards the second cathode 112 by the funnel 120. A voltage is applied to the second cathode 112 to generate a plasma discharge thereat.

The characteristics and magnitudes of the voltages applied to the first and second cathodes may be similar, but it may be advantageous for different duty periods to be employed for respective cathodes. This cathode arrangement with the second cathode downstream of the first seeks to maximise contact between the monatomic hydrogen/deuterium and the plasma and therefore the efficiency of the apparatus. This is further assisted by the funnel 120.

1. A method of releasing energy comprising the steps of providing an electrolyte having a catalyst therein, the catalyst being suitable for initiating transitions of hydrogen and/or deuterium atoms in the electrolyte to a sub-ground energy state, and being one of rubidium ions or potassium ions and having a concentration of between 1 mMol and 20 mMol, and generating a plasma discharge in the electrolyte, wherein the plasma discharge is generated by applying a voltage across electrodes in the electrolyte of between 50V and 20,000V.

2. The method of claim 1 wherein the voltage is applied so as to produce an intermittent plasma discharge.

3. The method of claim 1 wherein the applied voltage has a substantially square shaped waveform.

4. The method of claim 1 wherein the applied voltage has a pulsed waveform having a duty cycle between 0.001 and 0.5.

5. The method of claim 1 wherein the voltage is switched on and off by a switching assembly comprising an insulated gate bipolar transistor.

6. The method of claim 1 wherein the applied voltage has a waveform having a frequency of between DC and 100 kHz.

7. The method of claim 1 wherein a metal hydride is formed on an electrode which dissociates to form hydrogen and/or deuterium atoms.

8. The method of claim 1 wherein the metal hydride is formed on an electrode during voltage pulses and subsequently dissociates to form hydrogen and/or deuterium atoms.

9. The method of claim 1 wherein the current density generated by the applied voltage is 400,000 A/m² or above.

10. The method of claim 1 and further comprising the step of feeding the electrolyte past the electrodes.

11. The method of claim 1 and further comprising generating a magnetic field in the region of the electrodes.

12. The method of claim 2 wherein a cathode electrode comprises tungsten, zirconium, stainless steel, nickel and/or tantalum.

13. The method of claim 2 wherein the anode electrode is formed of a material which is inert with respect to the electrolyte.

14. The method of claim 16 wherein the anode comprises platinum, palladium and/or rhodium.

15. The method of claim 2 wherein the temperature of the plasma is approximately 6000K or above.

16. The method of claim 2 wherein the electrolyte comprises water and/or deuterated water and/or deuterium oxide.

17. The method of claim 21 wherein the only reactive ingredient consumed by the reaction is water or deuterated water.

18. The method of claim 2 and further comprising the step of heating the electrolyte to a temperature between 40 to 80° C. prior to generating the plasma discharge.

19. The method of claim 2 wherein fusion occurs via at least one of the following pathways:
²₁D+²₁D=³₂He+¹₀n
or
¹₁D+²₁D=³₁T+¹₁H
or
¹₁H+¹₁H=²₁D+β⁺+τ

20. A method of releasing energy comprising the steps of
providing an electrolyte having a catalyst therein, the catalyst being suitable for initiating transitions of hydrogen and/or deuterium atoms in the electrolyte to a sub-ground energy state and being capable of absorbing approximately (m*27.2)eV, where m is an integer, the catalyst being one of rubidium ions or potassium ions and having a concentration of between 1 mMol and 20 mMol, and
generating a plasma discharge in the electrolyte, wherein the plasma discharge is generated by applying a voltage across electrodes in the electrolyte of between 50V and 20,000V.
